(19) United States
(12) Patent Application Publication
(10) Pub. No.: US 2026/0275420 A1
(43) Pub. Date: Sep. 17, 2026

(54) METHOD FOR CONSTRUCTING MOLECULAR SUM GATES USING PADLOCK PROBES FOR QUANTITATING NUCLEIC ACID TARGETS SUMS

(71) Applicant: ALGORITHMIC BIOLOGICS PRIVATE LIMITED, BANGALORE (IN)

(72) Inventor: Manoj Gopalkrishnan, Mumbai (IN)

(21) Appl. No.: 19/167,001

(22) PCT Filed: Mar. 24, 2024
(86) PCT No.: PCT/IN2024/050309

§ 371 Date: Sep. 19, 2025

(30) Foreign Application Priority Data
Mar. 24, 2023 (IN) 202341021306
Mar. 24, 2024 (IN) 202444023087

(51) Int. Cl. C12Q 1/6886; C12Q 1/6811

(52) U.S. Cl. CPC C12Q 1/6886; C12Q 1/6811; C12Q 2600/156

(57) ABSTRACT

Embodiments herein provide a method for constructing molecular sum gates using padlock probes for quantitating the sum of concentrations of a large number of different nucleic acid targets species by (i) generating a molecular sum gate by introducing a plurality of padlock probes that are designed for binding to each target in a set of nucleic acid targets species (S), (ii) performing an assay in a testing device for quantitating the sum of concentrations of the set of nucleic acid targets species (S) in a sample by (a) obtaining one circularized padlock probes using a specific interaction of the padlock probes with a target nucleic acid, and (b) quantitating the sum of concentrations of the set of nucleic acid targets species (S) by means of quantification of the at least one circularized probe.

### BACKGROUND

### Technical Field

Embodiments herein generally relate to the analysis of a biological sample, and more particularly, to a method for constructing molecular sum gates using padlock probes for quantitating nucleic acid targets sums.

### Description of the Related Art

An assay is an analytical procedure used to measure the presence, amount, activity, or function of a molecule that determines the presence or absence of a substance or a target in a sample. Further, performing tests for each target separately increases the time and cost resources may be impractical.

Further, the existing methods for detecting a large number of targets becomes challenging. Additionally, the challenge lies in achieving high sensitivity and specificity while keeping the assay cost-effective and user-friendly for widespread application in research and diagnostics. Simultaneous detection of large number of targets in a single reaction can be complicated, leading to potential issues like decreased sensitivity, cross-reactivity, higher costs, and challenges in optimization and troubleshooting.

Therefore, there is a need to address the aforementioned technical drawbacks in existing technologies for method for constructing molecular sum gates using padlock probes for quantitating nucleic acid targets sums.

### SUMMARY

In view of the foregoing, according to a first aspect, there is provided a method for constructing molecular SUM gates using padlock probes for quantitating the sum of concentrations of a large number of different nucleic acid target species in a single reaction. The method includes (i) generating a molecular SUM gate by introducing a plurality of padlock probes that are designed for binding to each target region in a set of nucleic acid target species (S), wherein each of the plurality of padlock probes comprises a common region that enables detection of circularized probes and a variable region specific to a target nucleic acid, (ii) performing an assay in a testing device for quantitating the sum of concentrations of the set of nucleic acid targets species (S) in a sample by (a) obtaining at least one circularized padlock probe from the plurality of padlock probes using a specific interaction of the at least one padlock probe with a target nucleic acid associated with the at least one padlock probe, and (b) quantitating the sum of concentrations of the set of nucleic acid targets species (S) by means of quantification of the at least one circularized probe.

In some embodiments, the at least one circularized probes are quantitated using a real-time polymerase chain reaction (PCR), wherein the common region of the at least one circularized padlock probes hosts a PCR primer site and a plurality of hydrolysis probe binding sites that enable detection and quantification of the at least one circularized probes.

In some embodiments, the method comprises performing additional reactions to test individual expression levels of the at least one circularized padlock probes for identifying at least one nucleic acid target that exhibit the differential expression.

In some embodiments, the method implements a measurement channel in the testing device, which is based on a cycle threshold (Ct) value of the measurement channel, wherein the Ct value is generated using a quantitative PCR detection.

In some embodiments, the method comprises performing bulk transcriptomics of a large number of nucleic acid targets in a single reaction by detecting a significant change in the Ct value that indicates a differential expression of the at least nucleic acid target in response to a stimulus or a condition.

In some embodiments, differential expression patterns among the at least one nucleic acid targets are determined by comparing a first measurement obtained from a first sample to a second measurement obtained from a second sample, wherein the first sample is a control sample, and the second sample is subjected to a stimulus.

In some embodiments, the molecular SUM gate is employed to detect the presence or simultaneous absence of the set of nucleic acid targets species (S) by determining if the quantitation of the at least one circularized padlock probes is zero or nonzero.

In some embodiments, the molecular SUM gate is employed for cancer screening in a population by detecting the presence or absence of the set of nucleic acid target species (S).

In some embodiments, the molecular SUM gate is used for screening for BRCA mutations.

In some embodiments, the method further comprising detecting a sequence x using a means to detect a sequence y by making the set of nucleic acid target species (S) have exactly one target x and by configuring the common sequence of the padlock probe to be the sequence y.

In another aspect, there is provided a system for constructing molecular SUM gates using padlock probes for quantitating the sum of concentrations of a large number of different nucleic acid targets in a single reaction, the method comprising. The system comprises (i) a plurality of molecular SUM gates that are generated by introducing a plurality of padlock probes that are designed for binding to each target in a set of nucleic acid targets (S), wherein each of the plurality of padlock probes comprises a universal region specific to a target nucleic acid and a variable region that enables detection of circularized probes, and (ii) a testing device that is configured to perform an assay for quantitating the sum of concentrations of the set of nucleic acid targets species (S) in a sample by (a) obtaining at least one circularized padlock probe from the plurality of padlock probes using a specific interaction of the at least one padlock probe with a target nucleic acid associated with the at least one padlock probe, and quantitating the sum of concentrations of the set of nucleic acid targets species (S) by means of quantification of the at least one circularized probe.

In some embodiments, the molecular SUM gate is employed to detect the presence or simultaneous absence of the set of nucleic acid targets species (S) by determining if the quantitation of the at least one circularized padlock probes is zero or nonzero.

In some embodiments, the molecular SUM gate is employed for cancer screening in a population by detecting the presence or absence of the set of nucleic acid target species (S).

In some embodiments, the molecular SUM gate is employed for screening for BRCA mutations.

In some embodiments, the testing device further detects a sequence x using a means to detect a sequence y by making the set of nucleic acid targets (S) have exactly one target x and by configuring the common sequence of the padlock probe to be the sequence y.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed descriptions with reference to the drawings, in which:

FIG. 1A illustrates a system for identifying a presence or absence of a large number of different nucleic acid targets in reduced number of reactions using a molecular OR gate and a group testing method according to some embodiments herein;

FIG. 1B illustrates a system for constructing molecular SUM gates using padlock probes for quantitating the sum of concentrations of a large number of different nucleic acid target species in a single reaction according to some embodiments herein;

FIG. 2 is a block diagram that illustrates one or more modules of a pooling and decoding device of FIG. 1 according to some embodiments herein;

FIG. 3 illustrates an exemplary molecular inversion probe (MIP) for hybridizing a target sequence in a sample according to some embodiments herein;

FIGS. 4A-4C illustrate a gapless probe and a gapped probe for performing molecular inversion probe (MIP) based assay for detecting the plurality of targets in the sample according to some embodiments herein;

FIG. 5A illustrates a method for constructing molecular SUM gates using padlock probes for quantitating the sum of concentrations of a large number of different nucleic acid target species in a single reaction according to some embodiments herein;

FIG. 5B illustrates a method for detecting, identifying and quantitating a large number of targets in a sample using reduced assays according to some embodiments herein; and

FIG. 6 is a schematic diagram of a computer architecture of a pooling and decoding device or user device or testing device or a molecular computer or any computing device, in accordance with the embodiments herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

As mentioned, there remains a need for constructing molecular sum gates using padlock probes for quantitating nucleic acid targets sums. The embodiments herein achieve this by providing a system and method that enables constructing molecular sum gates using padlock probes for detecting large number of nucleic acid targets that includes the steps of (i) generating a molecular SUM gate by introducing a plurality of padlock probes that are designed for binding to each target region in a set of nucleic acid target species (S), wherein each of the plurality of padlock probes comprises a common region that enables detection of circularized probes and a variable region specific to a target nucleic acid, (ii) performing an assay in a testing device for quantitating the sum of concentrations of the set of nucleic acid targets species (S) in a sample by (a) obtaining at least one circularized padlock probe from the plurality of padlock probes using a specific interaction of the at least one padlock probe with a target nucleic acid associated with the at least one padlock probe, and (b) quantitating the sum of concentrations of the set of nucleic acid targets species (S) by means of quantification of the at least one circularized probe.

The term “sample” is referred to as may be a biological sample of a human being, plant, animal, bacterium, virus, fungi, or any other life form or a nonbiological sample. The sample may include, but is not limited to, a blood sample, a urine sample, a saliva sample, a swab sample, any biofluid or bodily fluid, any tissue sample, a tooth sample, a sweat sample, a nail sample, a skin sample, a hair sample, or a fecal sample, a leaf sample, a seed sample.

The term “nucleic acid targets” includes, but is not limited to, infectious agents or microbial analytes or disease-causing agents or pathogens, contamination agents, blood analytes, chemical species or chemical substances, proteins, nucleic acid sequences, alleles, marker regions and any biomolecules. The infectious agents may include, but not limited to, virus, bacteria, fungi, protozoa and helminth. The chemical species may include, but is not limited to, sodium (Na), potassium (K), urea, glucose, and creatinine. The chemical species or chemical substance is a substance that is composed of chemically identical molecular entities. The proteins are biomolecules comprised of amino acid residues joined together by peptide bonds. The protein may include, but is not limited to, antibodies, enzymes, hormones, transport proteins, and storage proteins. The nucleic acids include deoxyribonucleic acid (DNA), ribonucleic acid (RNA), or peptide nucleic acid (PNA), or synthetic nucleic acid analogues like Xeno Nucleic Acids. Biomolecules are any molecules that are produced by cells and living organisms.

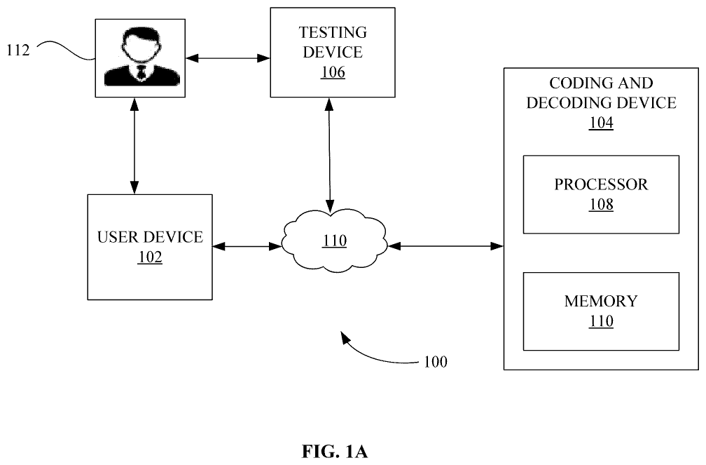

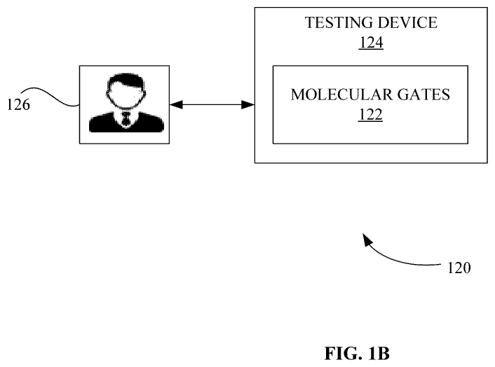

The embodiments herein allow detection, identification, and quantification of a large number of targets from a reduced number of tests, thus allowing the economy of both the biological sample and a number of tests. Referring now to the drawings and more particularly to FIGS. 1A through 6, where similar reference characters denote corresponding features consistently throughout the figures, there are shown preferred embodiments.

FIG. 1A illustrates a system 100 for detecting, identifying and quantitating a large number of targets in a sample using reduced number of assays according to some embodiments herein. The system 100 includes a user device 102, a coding and decoding device 104, and a testing machine 106 which are communicatively connected over a network 110. The coding and decoding device 104 includes a memory 110 that stores a set of instructions and a processor 108 that is configured to execute the set of instructions to perform one or more operations of the coding and decoding device 104. A user 112 may operate the testing device 106 and the user device 102. The coding and decoding device 104 may be a cloud computing device, a server, or a computing device. The cloud computing device may be a part of a public cloud or a private cloud. The server may be at least one of a standalone server, a server on a cloud, or the like. The computing device may include, but is not limited to, a personal computer, a notebook, a tablet, a desktop computer, a laptop, a handheld device, a mobile device, and the like. The coding and decoding device 104 may be at least one of, a microcontroller, a processor, a System on Chip (SoC), an integrated chip (IC), a microprocessor-based programmable consumer electronic device, and the like. The coding and decoding device 104 may communicate with an external entity through the network 110. The network 110 may be, but is not limited to, the Internet, a wired network, or a wireless network (a Wi-Fi network, a cellular network, a Wi-Fi Hotspot, Bluetooth, Zigbee, and the like).

FIG. 1B illustrates a system 120 for constructing molecular sum gates using padlock probes for detecting large number of nucleic acid targets. The system comprises a testing device 124 that implements molecular gates 122. A user 126 may operate the testing device 124.

The molecular gates 122 may be generated by introducing a plurality of padlock probes that are designed for binding to each target in a set of nucleic acid targets (S), wherein each of the plurality of padlock probes comprises a universal region specific to a target nucleic acid and a variable region that enables detection of circularized probes.

The testing device 124 may be configured to performing an assay for quantitating the sum of concentrations of the set of nucleic acid targets species (S) in a sample by (a) obtaining at least one circularized padlock probe from the plurality of padlock probes using a specific interaction of the at least one padlock probe with a target nucleic acid associated with the at least one padlock probe, and quantitating the sum of concentrations of the set of nucleic acid targets species (S) by means of quantification of the at least one circularized probe.

In some embodiments, the molecular SUM gate is employed to detect the presence or simultaneous absence of the set of nucleic acid targets species (S) by determining if the quantitation of the at least one circularized padlock probes is zero or nonzero.

In some embodiments, the molecular SUM gate is employed for cancer screening in a population by detecting the presence or absence of the set of nucleic acid target species (S).

In some embodiments, the molecular SUM gate is employed for screening for BRCA mutations.

In some embodiments, the testing device further detects a sequence x using a means to detect a sequence y by making the set of nucleic acid targets (S) have exactly one target x and by configuring the common sequence of the padlock probe to be the sequence y. The coding and decoding device 104 is configured to define a T-dimensional vector X of non-negative real numbers that denote an amount of each of the T targets in the sample to be tested, wherein true value of the T-dimensional vector X is unknown. The coding and decoding device 104 is configured to initiate a matrix B of size T×M, wherein M is a number of tests to be carried out and is determined using at least one of a logarithm scheme or a square root scheme.

The coding and decoding device 104 is configured to select columns of matrix B using a binary code logic that sets a k^{th} column of B to ‘1’ in a row n if and only if the k^{th} right shift of (n−1) ends with ‘1’, wherein each column of matrix B corresponds to a distinct subset of the T targets.

The coding and decoding device 104 is configured to perform M assays by implementing a molecular sum gate per each distinct subset of the T targets corresponding to the columns of matrix B, wherein output of each assay is equal to a summation of the amounts of targets in the corresponding column of matrix B. The coding and decoding device 104 is configured to obtain a measurement vector y of dimension M, wherein the measurement vector y is equal to a noisy version of (X*B), wherein the noisy version is obtained using a noise model characterized based on implementation of the molecular sum gate and based on the detection technology. The coding and decoding device 104 is configured to reconstruct the T-dimensional vector X using compressed sensing methods for detecting, identifying and quantitating a large number of targets in a sample using reduced assays.

In some embodiments, the coding and decoding device 104 may be used for Antibiotic Microbial Resistance (AMR) screening. AMR has become a leading cause of death worldwide with more than a million deaths per year. To treat antibiotic resistance requires knowing what antibiotic will be effective against the particular pathogen the patient is carrying. The current gold standard “antibiotic susceptibility test” provides this information, but it is very slow, taking several days to several weeks for a result. In comparison, Polymerase Chain Reaction (PCR) can be very fast and can even be delivered in a point-of-care fashion. However traditional PCR may miss antibiotic resistance markers since only a few targets can be targeted and detected in a few reactions. Running a comprehensive nucleic acid screen against hundreds of targets to identify which targets or antibiotic resistance mechanisms are present so that appropriate antibiotic susceptibility and resistance can be inferred requires much more expensive and non-point-of-care technologies like Next Generation Sequencing. The system 100 allows the identification of a large number of targets in a small number of reactions using a qPCR platform, thus allowing economy of time, economy of sample, and economy with respect to the number of tests required while using readily available qPCR machines.

In some embodiments, the system 100 may be used for cancer screening. Cancer screening can be done by taking a blood sample and looking for cell-free DNA markers indicating cancer. Such markers are known to exist, often in very low concentrations, at very early stages of several cancers. The advantage of such liquid biopsies is that they are minimally invasive, and they can be done for the population at large, for example as part of annual health checkups. However, if a liquid biopsy looks for only one cancer, the low prevalence of that single condition, the cost, and the inconvenience do not make a compelling health economics argument. On the other hand, if a liquid biopsy tries to offer a comprehensive screen with existing technology, the cost, turnaround time, or the amount of sample that needs to be collected to retain sensitivity makes the offering less attractive. Whereas, in the system 100, the possibility of a comprehensive liquid biopsy screen for a wide range of cancers that is highly sensitive is realized when many markers can be tested in a small number of reactions. Since one person is unlikely to be carrying a very large number of different cancers simultaneously, the vector X is sparse, and the methods are applicable. Since many cancers are being screened simultaneously, the health economics value of the test becomes much improved.

In some embodiments, the system 100 may be used for liquid biopsies for immune rejection: A liquid biopsy is a blood sample in which we look for nucleic acid markers indicating after organ transplants. Such markers are known to exist, often in very low concentrations, in the process of immune rejection. The advantage of liquid biopsies is that they are minimally invasive and provide early detection of immune rejection so that steroid doses can be carefully calibrated. However, if a liquid biopsy looks for only one mechanism of immune rejection, the cost and the inconvenience do not make a compelling health economics argument. If a liquid biopsy tries to offer a comprehensive screen, the cost, turnaround time, and amount of sample that needs to be collected to retain sensitivity become infeasible. Whereas in the system 100, the possibility of a comprehensive liquid biopsy screen that is highly sensitive is realized when many markers can be tested in a small number of reactions. The assumption of low prevalence will be justified since one person is unlikely to be carrying a very large number of immune rejection mechanisms simultaneously. This can be a game changer for how we treat Organ Transplant Rejections.

In some embodiments, the system 100 is used for single nucleotide polymorphism panels. Genotyping means taking a genome and classifying it into one of many types. Often the types are determined by which nucleotide pair is present at multiple particular locations on a genome. Such variations are known as Single Nucleotide Polymorphisms or SNPs. It is often the case that at any particular position one allele is much more frequent than the other alleles. This allele is then called the reference allele for that position, and the differing alleles are called mutant alleles. So for example 100 locations (or loci) may be typed, and a particular sample may have a mutant allele on a small number (<10) of these locations. Currently genotyping for SNPs can be done with PCR for a few locations (1-10), or with microarrays for a larger number (1000-10,000) or with sequencing for even larger number of locations. SNP panels are important in medical diagnosis, personalized medicine, even in cancer therapeutics. They are also important in molecular breeding in agriculture. Whereas, the system 100 allows such SNP panels to be performed with PCR for several hundred loci simultaneously with economy in cost and frugal use of sample.

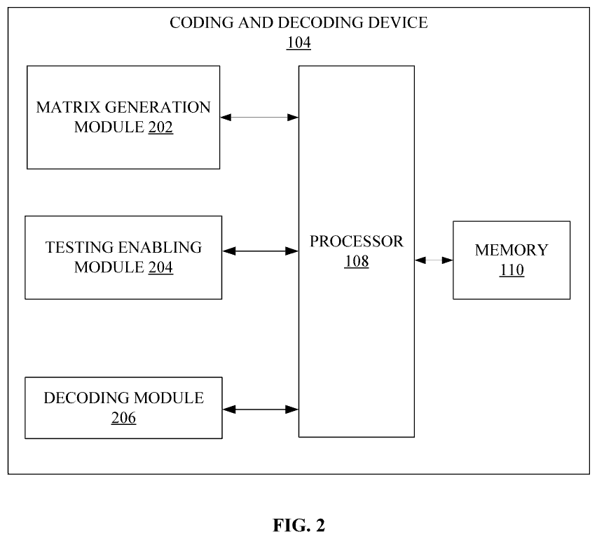

FIG. 2 is a block diagram that illustrates one or more modules of the coding and decoding device 104 of FIG. 1 according to some embodiments herein. The coding and decoding device 104 includes a matrix generation module 202, a testing enabling module 204, a decoding module 206 that are connected to the processor 108 that is connected with the memory 110. In some embodiments, the coding and decoding device 104 may include an embedded database to store information related to sample analysis.

The matrix generation module 202 defines a T-dimensional vector X of non-negative real numbers that denote an amount of each of the T targets in the sample to be tested, wherein true value of the T-dimensional vector X is unknown. In some embodiments, the T-dimensional vector X has a regular structure allowing for the possibility of compression. The matrix generation module 202 initiates a matrix B of size T×M, wherein M is a number of tests to be carried out and is determined using at least one of a logarithm scheme or a square root scheme. In some embodiments, the logarithm scheme includes (2*┌log 2T┐) and the square root scheme includes (3*┌√T┐).

The matrix generation module 202 selects columns of matrix B using a binary code logic that sets a k^{th} column of B to ‘1’ in a row n if and only if the k^{th} right shift of (n−1) ends with ‘1’, wherein each column of matrix B corresponds to a distinct subset of the T targets. For example, a column j of the matrix B corresponds to a subset S_j⊂T, where T is the set of all targets and for each subset S_j, there is a molecular sum gate SUM(S_j).

In some embodiments, the columns of the matrix B are selected based on the following pseudo code:

```
def generate_binary_arr(m):
  arr = [0 for i in range(8)]
  res = m
  for k in range(7,−1,−1):
    if res >= 2**k:
      res = res − 2**k
      arr[k] = 1
    if res == 0:
      break
  return arr
def create_B(n, m):
  # Initialize an empty matrix B
  B = np.zeros((n, m), dtype=int)
  #Loop over all the elemente
  for k in range(m):
    #Generate the binary Matrix
    arr = generate_binary_arr(k)
    #Fill the row of the corresponding elements
    for i,val in enumerate(arr):
      if val == 0:
        B[2*i,k] = 1
      else:
        B[2*i + 1,k] = 1
return B
```

The matrix B may contain the following entries:

$$[ 1010101010101010 ]$$

$$[ 0110101010101010 ]$$

$$[ 1001101010101010 ]$$

$$[ 0101101010101010 ]$$

$$⋮$$

$$[ 1010010101010101 ]$$

$$[ 0110010101010101 ]$$

$$[ 1001010101010101 ]$$

$$[ 0101010101010101 ]$$

For example, row 37^{th} of the matrix B is [1 0 1 0 0 1 1 0 1 0 0 1 1 0 1 0].

The testing enabling module 204 enables a user to perform M assays by implementing a molecular sum gate per each distinct subset of the T targets corresponding to the columns of matrix B, wherein output of each assay is equal to a summation of the amounts of targets in the corresponding column of matrix B. The testing enabling module 204 is configured to obtain a measurement vector y of dimension M, wherein the measurement vector y is equal to a noisy version of (X*B), wherein the noisy version is obtained using a noise model characterized based on implementation of the molecular sum gate. Molecular sum gates are a type of DNA-based molecular computing component that can perform arithmetic operations, specifically addition.

In some embodiments, the molecular sum gate is implemented using a first construction that pools primers for the different targets s∈S and performs a qPCR assay to achieve a molecular sum gate SUM(S). However, this construction suffers from a problem of unintended cross-reactivity that may lead to false amplifications and poor accuracy of the assay when the cardinality of S becomes large. In some embodiments, the molecular sum gate is implemented using a second construction that addresses the problem of the first construction by using padlock probes or molecular inversion probes that are designed to bind specifically to target regions and are amenable to large scale multiplexing. In the second construction, one padlock probe is introduced for each target s∈S, and all the padlock probes share a universal linker region which may be detected and quantified using qPCR.

The decoding module 206 is configured perform reconstruction of the T-dimensional vector X using compressed sensing methods for detecting, identifying and quantitating a large number of targets in a sample using reduced assays.

In some embodiments, the process is applied to any number of targets by modifying the size of the B matrix to match the number of targets. Given a set of N targets, a B matrix of size N×m can be constructed, where m is the number of tests to be carried out.

In an example scenario, let B be a matrix of size 256×16 where n=256 represents the number of targets to be detected in one sample and m=16 is the number of tests to be carried out to achieve this detection. Such a matrix B has to be chosen carefully. One option for choosing B comes from the binary code which we now describe to fix ideas. For example, we can write all numbers from 0 to 255 as 8-bit strings of zeros and ones. For example, a string of binary numbers “10110101” corresponds to the number 181. The k^{th} right shift of the string is defined as the string obtained by omitting the k rightmost characters. For example, the 2^{nd} right shift of “10110101” is “101101”. The parity of a string is odd if and only if the string end with “1”, and it is even otherwise. Therefore, the second right shift of 181 has odd parity.

If C1, C2, . . . , C16 are the columns of the pooling matrix B, each column is a vector of 256 dimensions. The columns can be chosen as per the following logic for rows n=1 to 256:
C1 has a 1 in row n if and only if (n−1) is odd, else it has a 0
C2 has a 1 in row n if and only if (n−1) is even, else it has a 0
C3 has a 1 in row n if and only if the 1^{st} right shift of (n−1) is odd
C4 has a 1 in row n if and only if the 1^{st} right shift of (n−1) is even
C5 has a 1 in row n if and only if the 2^{nd} right shift of (n−1) is odd
C6 has a 1 in row n if and only if the 2^{nd} right shift of (n−1) is even
   Similarly C7 and C8 are decided by the parity of the 3^{rd} right shift, C9 and C10 by the parity of the 4^{th} right shift, C11 and C12 by the parity of the 5^{th} right shift, C13 and C14 by the parity of the 6th right shift, and finally C15 and C16 by the parity of the 7th right shift, which is the same as the parity of the most significant bit, or whether the number is less than 128 or greater than or equal to. It is verified that the 182^{nd} row in C5 contains a value “1” and in C6 contains a value “0”. Given an appropriate matrix B, column j of B corresponds to a subset S_j⊂T. Corresponding to this subset there is a molecular sum gate SUM(Sj) whose output is available.

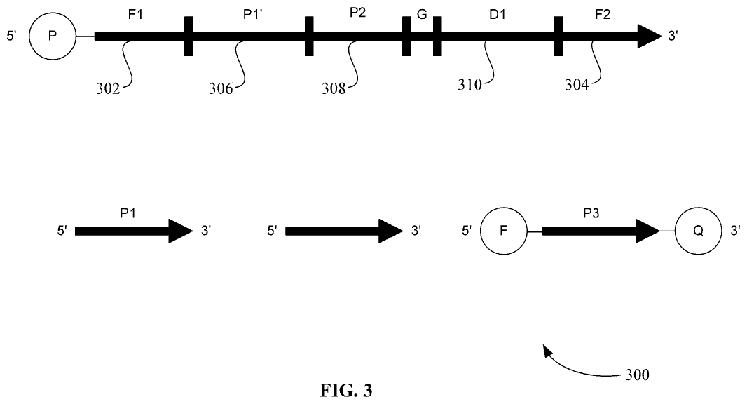

FIG. 3 illustrates an exemplary molecular inversion probe (MIP) 300 for hybridizing a target sequence in a sample, according to some embodiments herein. The MIP 300 includes a first flanking region (F1) 302 at a 5′ end, a second flanking region (F2) 304 at the 3′ end, a first primer (P1′) 306, a second primer (P2) 308, and a detection sequence (D1) 310. The first flanking region (F1) 302 and second flanking region (F2) 304 comprise sequences that are complementary to the target sequence present in the sample. The first flanking region (F1) 302 and second flanking region (F2) 304 may vary from probe to probe. The first primer (P1′) 306, second primer (P2) 308, and detection sequence (D1) 310 are common to all probes. The first primer (P1′) 306, second primer (P2) 308, and third primer (P3) are universal primers.

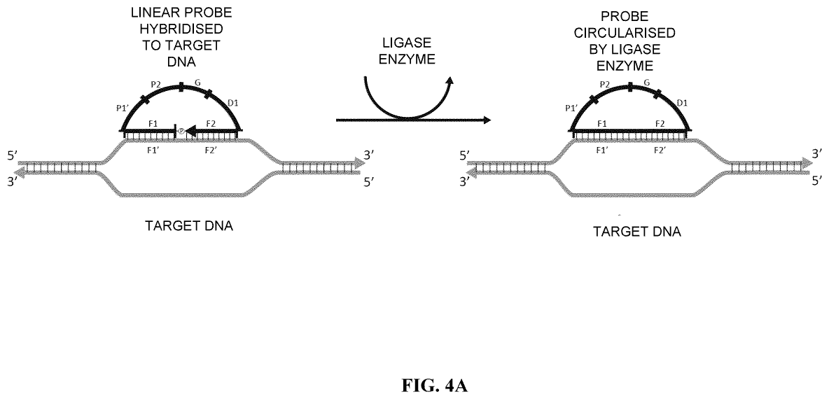

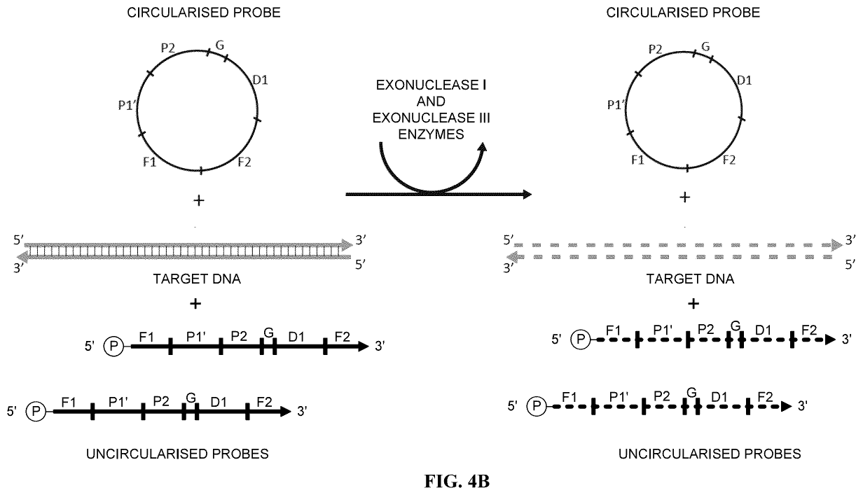

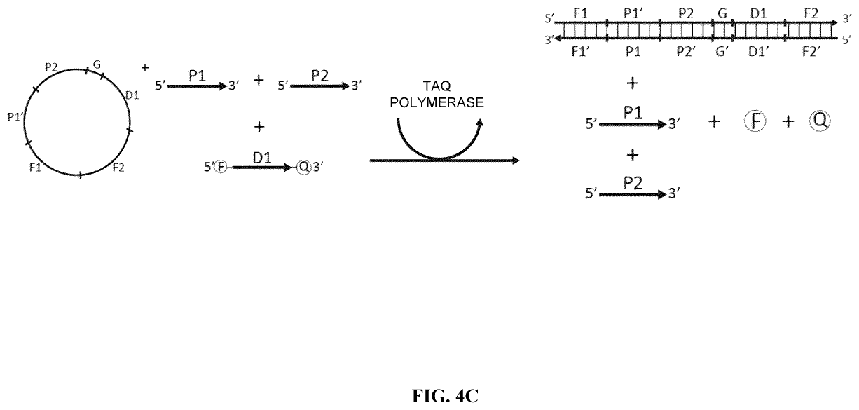

FIGS. 4A-4C illustrate a gapless probe and a gapped probe for performing molecular inversion probe (MIP) based assay for detecting the plurality of targets in the sample, according to some embodiments herein. FIG. 4A illustrates probe hybridization and ligation. Linear molecular inversion probe hybridizes to target DNA and is circularized by a ligase enzyme. FIG. 4B illustrates exonuclease mediated selection. Uncircularised probes and target DNA are degraded by the action of exonuclease enzymes. FIG. 4C illustrates qPCR based detection of circularized probes. Circularised probes are detected in a qPCR reaction using fluorescence based probes. The gapless probe is a probe that hybridizes with a target sequence without gaps between a first flanking region and a second flanking region, thereafter the first flanking region and second flanking region are ligated. For the gapped probe, an extension is done to seal gap between flanks before ligation.

After probe hybridization and ligation, exonuclease degradation of uncircularised probes is performed, as illustrated in FIG. 4B. Exonucleases are used to degrade linear DNA templates and unbound probes. Subsequently, circularized probes are amplified and detected using qPCR, as illustrated in FIG. 4C.

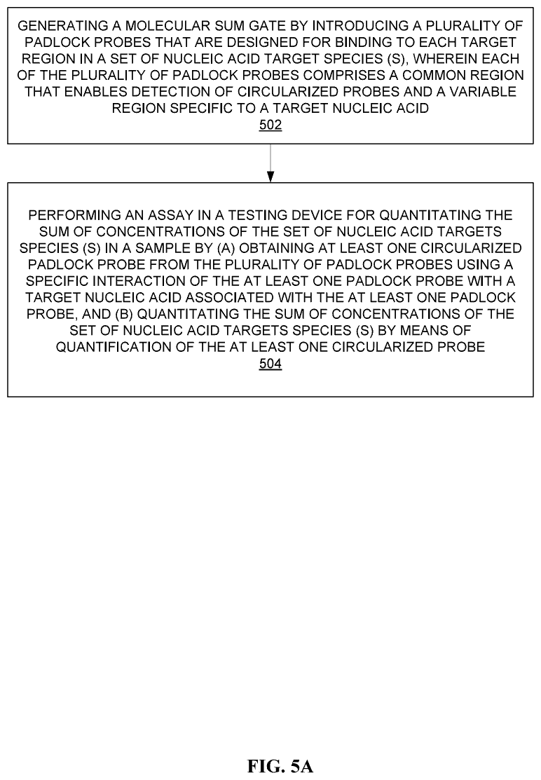

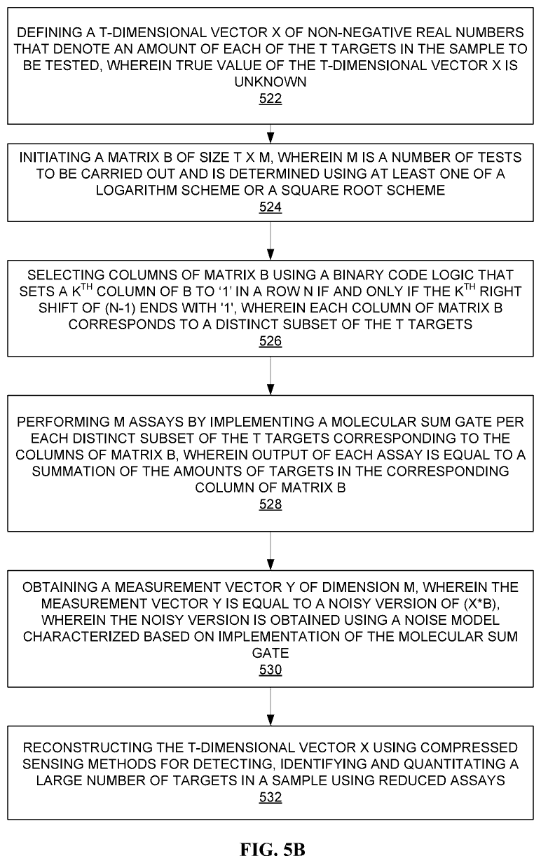

FIG. 5A illustrates a method for constructing molecular SUM gates using padlock probes for quantitating the sum of concentrations of a large number of different nucleic acid target species in a single reaction. At step 502, the method includes generating a molecular SUM gate by introducing a plurality of padlock probes that are designed for binding to each target region in a set of nucleic acid target species (S), wherein each of the plurality of padlock probes comprises a common region that enables detection of circularized probes and a variable region specific to a target nucleic acid. At step 504, the method includes performing an assay in a testing device for quantitating the sum of concentrations of the set of nucleic acid targets species (S) in a sample by (a) obtaining at least one circularized padlock probe from the plurality of padlock probes using a specific interaction of the at least one padlock probe with a target nucleic acid associated with the at least one padlock probe, and (b) quantitating the sum of concentrations of the set of nucleic acid targets species (S) by means of quantification of the at least one circularized probe.

In some embodiments, the at least one circularized probes are quantitated using a real-time polymerase chain reaction (PCR), wherein the common region of the at least one circularized padlock probes hosts a PCR primer site and a plurality of hydrolysis probe binding sites that enable detection and quantification of the at least one circularized probes.

In some embodiments, the method comprises performing additional reactions to test individual expression levels of the at least one circularized padlock probes for identifying at least one nucleic acid target that exhibit the differential expression.

In some embodiments, the method implements a measurement channel in the testing device, which is based on a cycle threshold (Ct) value of the measurement channel, wherein the Ct value is generated using a quantitative PCR detection.

In some embodiments, the method comprises performing bulk transcriptomics of a large number of nucleic acid targets in a single reaction by detecting a significant change in the Ct value that indicates a differential expression of at least nucleic acid target in response to a stimulus or a condition.

In some embodiments, differential expression patterns among the at least one nucleic acid targets are determined by comparing a first measurement obtained from a first sample to a second measurement obtained from a second sample, wherein the first sample is a control sample, and the second sample is subjected to a stimulus.

In some embodiments, the molecular SUM gate is employed to detect the presence or simultaneous absence of the set of nucleic acid targets species (S) by determining if the quantitation of the at least one circularized padlock probes is zero or nonzero.

In some embodiments, the molecular SUM gate is employed for cancer screening in a population by detecting the presence or absence of the set of nucleic acid target species (S).

In some embodiments, the molecular SUM gate is employed for screening for BRCA mutations.

In some embodiments, the method further comprises detecting a sequence x using a means to detect a sequence y by making the set of nucleic acid targets (S) have exactly one target x and by configuring the common sequence of the padlock probe to be the sequence y.

The below-mentioned experiment design demonstrates the behaviour of the sum gate. The sum gate can detect multiple targets in a single pool (detected on the same channel). The output qPCR reports a Ct value that is commensurate with the sum of the amounts of those targets.

The 54-probe pool utilized in the preceding section has been repurposed for the experiments detailed in this section. The FAM channel targets are employed to showcase sum gate behavior in the experiment. The three distinct targets were utilized, testing reactions for each target individually, in combinations of two targets, and with all three targets simultaneously. A quantity of 5*10\hat{ }6 copies of each target was employed in these experiments. As a result, the table below shows the Ct value obtained when testing for targets 1, 2 and 3 individually, combinations of 2 targets among targets 1, 2 and 3 and all three targets simultaneously. The data clearly shows that the Ct1 target>Ct 2 targets>Ct 3 targets as expected when testing for multiple targets on the same channel simultaneously.

| Target | Ct value |
|---|---|
| 1 | 25.294 |
| 2 | 25.207 |
| 3 | 25.315 |
| 1 + 2 | 24.711 |
| 1 + 3 | 24.549 |
| 2 + 3 | 24.340 |
| 1 + 2 + 3 | 23.178 |
| No Targets | No Ct |

The table “Table 1” demonstrates that compressed PCR allows testing for a large number of targets in a few reactions. The compressed CPR detects 81 different targets through 9 reactions. The targets may be DNA targets, RNA targets, PNA targets, and nucleic acid. The fluorescent channels may be a FAM channel, a HEX channel, and a CY5 channel. In the experiment, the output of the reactions is recorded as qPCR cT values across the fluorescent channels. Each reaction test for 27 unique DNA targets and each target is tested for three times across all 9 reactions. The experiment demonstrates that on the entire panel of 9 reactions using a single pool of 27 padlock probes, 27 unique DNA targets are tested in a single reaction. The pool, which tests for 27 targets, has been used to simulate the entire panel and has been run 9 times for each experimental run. Positive targets have been simulated using multiple different synthetic oligonucleotides that serve as targets for different padlock probes in our pool. Table 1 illustrates the panel simulated in this experiment. Targets are numbered 1-81, and the fluorescent channel column shows targets detected by fluorophores FAM, BEX, and CY5. All of our data have been generated using Reaction 1, which tests for 27 targets multiplexed into a single reaction across three fluorescent channels as shown below. In this reaction, targets 1-9 are detected on the FAM channel, targets 10-18 are detected on the BEX channel, and targets 19-27 are detected on the CY5 channel.

TABLE 1

| FLUORESCENT | REAC- | REAC- | REAC- | REAC- | REAC- | REAC- | REAC- | REAC- | REAC- |
|---|---|---|---|---|---|---|---|---|---|
| CHANNEL | TION-1 | TION-2 | TION-3 | TION-4 | TION-5 | TION-6 | TION-7 | TION-8 | TION-9 |
| FAM | 1 | 28 | 55 | 1 | 4 | 7 | 1 | 4 | 7 |
| | 2 | 29 | 56 | 10 | 13 | 16 | 11 | 14 | 17 |
| | 3 | 30 | 57 | 19 | 22 | 25 | 21 | 24 | 27 |
| | 4 | 31 | 58 | 28 | 31 | 34 | 31 | 34 | 28 |
| | 5 | 32 | 59 | 37 | 40 | 43 | 41 | 44 | 38 |
| | 6 | 33 | 60 | 46 | 49 | 52 | 51 | 54 | 48 |
| | 7 | 34 | 61 | 55 | 58 | 61 | 61 | 55 | 58 |
| | 8 | 35 | 62 | 64 | 67 | 70 | 71 | 65 | 68 |
| | 9 | 36 | 63 | 73 | 76 | 79 | 81 | 75 | 78 |
| HEX | 10 | 37 | 64 | 2 | 5 | 8 | 2 | 5 | 8 |
| | 11 | 38 | 65 | 11 | 14 | 17 | 12 | 15 | 18 |
| | 12 | 39 | 66 | 20 | 23 | 26 | 22 | 25 | 19 |
| | 13 | 40 | 67 | 29 | 32 | 35 | 32 | 35 | 29 |
| | 14 | 41 | 68 | 38 | 41 | 44 | 42 | 45 | 39 |
| | 15 | 42 | 69 | 47 | 50 | 53 | 52 | 46 | 49 |
| | 16 | 43 | 70 | 56 | 59 | 62 | 62 | 56 | 59 |
| | 17 | 44 | 71 | 65 | 68 | 71 | 72 | 66 | 69 |
| | 18 | 45 | 72 | 74 | 77 | 80 | 73 | 76 | 79 |
| CY5 | 19 | 46 | 73 | 3 | 6 | 9 | 3 | 6 | 9 |
| | 20 | 47 | 74 | 12 | 15 | 18 | 13 | 16 | 10 |
| | 21 | 48 | 75 | 21 | 24 | 27 | 23 | 26 | 20 |
| | 22 | 49 | 76 | 30 | 33 | 36 | 33 | 36 | 30 |
| | 23 | 50 | 77 | 39 | 42 | 45 | 43 | 37 | 40 |
| | 24 | 51 | 78 | 48 | 51 | 54 | 53 | 47 | 50 |
| | 25 | 52 | 79 | 57 | 60 | 63 | 63 | 57 | 60 |
| | 26 | 53 | 80 | 66 | 69 | 72 | 64 | 67 | 70 |
| | 27 | 54 | 81 | 75 | 78 | 81 | 74 | 77 | 80 |

Table 2 demonstrates that each reaction serves as a molecular SUM gate, reporting on the presence or absence of 27 different targets simultaneously. The output of these reactions is analyzed by an algorithm to identify all targets present in E the sample. The algorithm categorizes each target as positive (indicating its presence in the sample), negative (indicating its absence in the sample), or undetermined (suggesting that particular target may be present in the sample and necessitates re-testing). Below is a summary of the results from 5 different experiments with varying numbers of positives. Across all 5 experiments, we reported 0 false negatives, 1 false positive, and 1 undetermined target that required re-testing.

| | | Preva- | | | | | | | |
|---|---|---|---|---|---|---|---|---|---|
| Experi- | | lance | True | False | True | False | Sensi- | Speci- | Undetermined |
| ments | Positive | rate | Positive | Negative | Negative | Positive | tivity | ficity | target |
| Trial 1 | 1 | 1.23% | 1 | 0 | 80 | 0 | 100.00% | 100.00% | 0 |
| Trial 2 | 2 | 2.47% | 2 | 0 | 79 | 0 | 100.00% | 100.00% | 0 |
| Trial 3 | 3 | 3.70% | 3 | 0 | 78 | 0 | 100.00% | 100.00% | 0 |
| Trial 4 | 4 | 4.94% | 4 | 0 | 76 | 0 | 100.00% | 100.00% | 0 |
| Trial 5 | 5 | 6.17% | 5 | 0 | 75 | 0 | 100.00% | 100.00% | 1 |

Trial-1 data:

| Reaction | FAM | HEX | CY5 | Internal control |
|---|---|---|---|---|
| 1 | 30.252 | No ct | No ct | 33.723 |
| 2 | No ct | No ct | No ct | 34.758 |
| 3 | No ct | No ct | No ct | 35.425 |
| 4 | No ct | No ct | No ct | 34.685 |
| 5 | 30.642 | No ct | No ct | 33.402 |
| 6 | No ct | No ct | No ct | 35.018 |
| 7 | No ct | No ct | No ct | 34.538 |
| 8 | 30.823 | No ct | No ct | 33.503 |
| 9 | No ct | No ct | No ct | 34.328 |

| Positive predicted by Tapestry | Ground Truth |
|---|---|
| 4 | 4 |

Trial-2 data:

| Reaction | FAM | HEX | CY5 | Internal control |
|---|---|---|---|---|
| 1 | No ct | 31.530 | No ct | 32.645 |
| 2 | No ct | No ct | No ct | 34.963 |
| 3 | No ct | 31.450 | No ct | 32.433 |
| 4 | No ct | No ct | No ct | 33.603 |
| 5 | No ct | No ct | 33.794 | 33.656 |
| 6 | No ct | No ct | 34.108 | 33.504 |
| 7 | No ct | 31.683 | No ct | 33.016 |
| 8 | No ct | 31.368 | No ct | 30.549 |
| 9 | No ct | No ct | No ct | 35.002 |

| Positive predicted by Tapestry | Ground Truth |
|---|---|
| 15, 72 | 15, 72 |

Trial-3 data:

| Reaction | FAM | HEX | CY5 | Internal control |
|---|---|---|---|---|
| 1 | 30.258 | No ct | 31.737 | 32.358 |
| 2 | No ct | No ct | No ct | 34.397 |
| 3 | 31.551 | No ct | No ct | 34.077 |
| 4 | No ct | 39.964 | No ct | 34.139 |
| 5 | No ct | 30.912 | No ct | 32.241 |
| 6 | 31.396 | No ct | 32.521 | 32.647 |
| 7 | 32.760 | No ct | 34.276 | 33.273 |
| 8 | No ct | No ct | No ct | 33.857 |
| 9 | No ct | No ct | 32.300 | 33.211 |

| Positive predicted by Tapestry | Ground Truth |
|---|---|
| 9, 23, 61 | 9, 23, 61 |

Trial-4 data:

| Reaction | FAM | HEX | CY5 | Internal control |
|---|---|---|---|---|
| 1 | No ct | 27.063 | No ct | 20.590 |
| 2 | 25.767 | No ct | No ct | 20.646 |
| 3 | No ct | 25.857 | 26.968 | 20.818 |
| 4 | 25.594 | 26.549 | No ct | 20.910 |
| 5 | 25.375 | No ct | No ct | 20.729 |
| 6 | No ct | 26.341 | No ct | 20.246 |
| 7 | No ct | No ct | 26.307 | 20.270 |
| 8 | No ct | 26.815 | No ct | 20.650 |
| 9 | 24.970 | 26.006 | No ct | 20.750 |

| Positive predicted by Tapestry | Ground Truth |
|---|---|
| 17, 64, 29, 76 | 17, 64, 29, 76 |

Trial-5 data:

| Reaction | FAM | HEX | CY5 | Internal control |
|---|---|---|---|---|
| 1 | No ct | 26.240 | 26.042 | 20.122 |
| 2 | No ct | 26.723 | No ct | 20.443 |
| 3 | 24.945 | No ct | 25.630 | 20.186 |
| 4 | No ct | No ct | 26.430 | 20.637 |
| 5 | 24.715 | No ct | 26.054 | 19.924 |
| 6 | 23.788 | No ct | No ct | 20.041 |
| 7 | No ct | 25.292 | 24.699 | 20.085 |
| 8 | 23.831 | No ct | No ct | 19.951 |
| 9 | 24.634 | 26.887 | No ct | 20.620 |

Tapestry Prediction

| Positive | Undetermined | Ground Truth |
|---|---|---|
| 12, 24, 58, 79, 43 | 75 | 12, 24, 58, 79, 43 |

FIG. 5B illustrates a method for detecting, identifying, and quantitating a large number of targets in a sample using reduced number of assays according to some embodiments herein. At step 522, the method includes defining a T-dimensional vector X of nonnegative real numbers that denote an amount of each of the T targets in the sample to be tested, wherein true value of the T-dimensional vector X is unknown and may have sparse support such that most of the entries are zero. At step 524, the method includes initiating a matrix B of size T×M, wherein M is a number of tests to be carried out and is determined using at least one of a logarithm scheme or a square root scheme. At step 526, the method includes selecting columns of matrix B using a binary code logic that sets a k^{th} column of B to ‘1’ in a row n if and only if the k^{th} right shift of (n−1) ends with ‘1’, wherein each column of matrix B corresponds to a distinct subset of the T targets. At step 528, the method includes performing M assays by implementing a molecular sum gate per each distinct subset of the T targets corresponding to the columns of matrix B, wherein output of each assay is equal to a summation of the amounts of targets in the corresponding column of matrix B. At step 530, the method includes obtaining a measurement vector y of dimension M, wherein the measurement vector y is equal to a noisy version of (X*B), wherein the noisy version is obtained using a noise model characterized based on implementation of the molecular sum gate. At step 532, the method includes reconstructing the T-dimensional vector X using compressed sensing methods for detecting, identifying and quantitating a large number of targets in a sample using reduced assays by making use of the regularity conditions like sparsity on X.

The method is particularly applicable in situations where a low prevalence assumption holds. In such settings, only a small percentage of targets can be present simultaneously in the sample. Hence, it is assumed that the T-dimensional vector X has sparse support, i.e., most of the entries are zero.

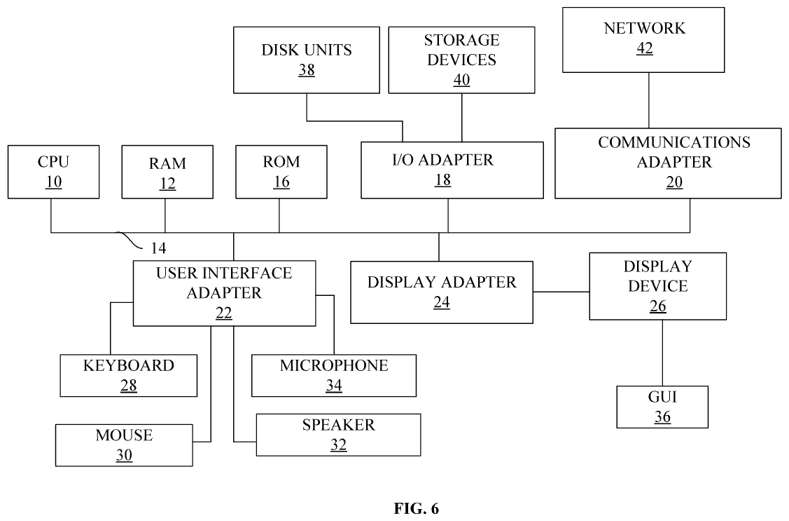

FIG. 6 is a schematic diagram of computer architecture of a pooling and decoding device or user device or testing device or a molecular computer or a computing device, in accordance with the embodiments herein. A representative hardware environment for practicing the embodiments herein is depicted in FIG. 6, with reference to FIGS. 1 through 5. This schematic drawing illustrates a hardware configuration of a server/computer system/computing device in accordance with the embodiments herein. The system 100 includes at least one processing device CPU 10 that may be interconnected via system bus 14 to various devices such as a random-access memory (RAM) 12, read-only memory (ROM) 16, and an input/output (I/O) adapter 18. The I/O adapter 18 can connect to peripheral devices, such as disk units 38 and program storage devices 40 that are readable by the system. The system can read the inventive instructions on the program storage devices 40 and follow these instructions to execute the methodology of the embodiments herein. The system further includes a user interface adapter 22 that connects a keyboard 28, mouse 30, speaker 32, microphone 34, and/or other user interface devices such as a touch screen device (not shown) to the bus 14 to gather user input. Additionally, a communication adapter 20 connects the bus 14 to a data processing network 42, and a display adapter 24 connects the bus 14 to a display device 26, which provides a graphical user interface (GUI) 36 of the output data in accordance with the embodiments herein, or which may be embodied as an output device such as a monitor, printer, or transmitter, for example.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents employed herein is for the purpose of description and not of limitation. Therefore, while the embodiments herein have been described in terms of preferred embodiments, those skilled in the art will recognize that the embodiments herein can be practiced with modification within the scope.

What is claimed is:

1. A method for constructing molecular SUM gates using padlock probes for quantitating the sum of concentrations of a large number of different nucleic acid target species in a single reaction, the method comprising:
characterized in that, generating a molecular SUM gate by introducing a plurality of padlock probes that are designed for binding to each target region in a set of nucleic acid target species (S), wherein each of the plurality of padlock probes comprises a common region that enables detection of circularized probes and a variable region specific to a target nucleic acid;
performing an assay in a testing device for quantitating the sum of concentrations of the set of nucleic acid targets species (S) in a sample by:
obtaining at least one circularized padlock probe from the plurality of padlock probes using a specific interaction of the at least one padlock probe with a target nucleic acid associated with the at least one padlock probe, and
quantitating the sum of concentrations of the set of nucleic acid targets species (S) by means of quantification of the at least one circularized probe.

2. The method as claimed in claim 1, wherein the at least one circularized probes are quantitated using a real-time polymerase chain reaction (PCR), wherein the common region of the at least one circularized padlock probes hosts a PCR primer site and a plurality of hydrolysis probe binding sites that enable detection and quantification of the at least one circularized probes.

3. The method as claimed in claim 1, wherein the method comprises performing additional reactions to test individual expression levels of the at least one circularized padlock probes for identifying at least one nucleic acid target that exhibit the differential expression.

4. The method as claimed in claim 1, wherein the method implements a measurement channel in the testing device, which is based on a cycle threshold (Ct) value of the measurement channel, wherein the Ct value is generated using a quantitative PCR detection.

5. The method as claimed in claim 4, wherein the method comprises performing bulk transcriptomics of a large number of nucleic acid targets in a single reaction by detecting a significant change in the Ct value that indicates a differential expression of the at least nucleic acid target in response to a stimulus or a condition.

6. The method as claimed in claim 5, wherein the differential expression patterns among the at least one nucleic acid targets are determined by comparing a first measurement obtained from a first sample to a second measurement obtained from a second sample, wherein the first sample is a control sample, and the second sample is subjected to a stimulus.

7. The method as claimed in claim 1, wherein the molecular SUM gate is employed to detect the presence or simultaneous absence of the set of nucleic acid targets species (S) by determining if the quantitation of the at least one circularized padlock probes is zero or nonzero.

8. The method as claimed in claim 1, wherein the molecular SUM gate is employed for cancer screening in a population by detecting the presence or absence of the set of nucleic acid target species (S).

9. The method as claimed in claim 8, wherein the molecular SUM gate is employed for screening for BRCA mutations.

10. The method as claimed in claim 1, the method further comprises detecting a sequence x using a means to detect a sequence y by making the set of nucleic acid targets species (S) have exactly one target x and by configuring the common sequence of the padlock probe to be the sequence y.

11. A system for constructing molecular SUM gates using padlock probes for quantitating the sum of concentrations of a large number of different nucleic acid targets in a single reaction, the method comprising, the system comprising:
characterized in that;
a plurality of molecular SUM gates (122) that are generated by introducing a plurality of padlock probes that are designed for binding to each target in a set of nucleic acid targets (S), wherein each of the plurality of padlock probes comprises a universal region specific to a target nucleic acid and a variable region that enables detection of circularized probes;
a testing device (124) that is configured to perform an assay for quantitating the sum of concentrations of the set of nucleic acid targets species (S) in a sample by:
obtaining at least one circularized padlock probe from the plurality of padlock probes using a specific interaction of the at least one padlock probe with a target nucleic acid associated with the at least one padlock probe, and
quantitating the sum of concentrations of the set of nucleic acid targets species (S) by means of quantification of the at least one circularized probe.

12. The system as claimed in claim 11, wherein the molecular SUM gate is employed to detect the presence or simultaneous absence of the set of nucleic acid targets species (S) by determining if the quantitation of the at least one circularized padlock probes is zero or nonzero.

13. The system as claimed in claim 11, wherein the molecular SUM gate is employed for cancer screening in a population by detecting the presence or absence of the set of nucleic acid target species (S).

14. The system as claimed in claim 13, wherein the molecular SUM gate is employed for screening for BRCA mutations.

15. The system as claimed in claim 10, wherein the testing device (124) further detects a sequence x using a means to detect a sequence y by making the set of nucleic acid targets species (S) have exactly one target x and by configuring the common sequence of the padlock probe to be the sequence y.
